(19) United States
(12) Patent Application Publication
(10) Pub. No.: US 2023/0054407 A1
(43) Pub. Date: Feb. 23, 2023

(54) OPTICAL BIOPSY STAIN PANELS AND METHODS OF USE

(71) Applicant: ARIZONA BOARD OF REGENTS ON BEHALF OF THE UNIVERSITY OF ARIZONA, Tucson, AZ (US)

(72) Inventors: Michael C. Larson, Tucson, AZ (US); Urs Utzinger, Tucson, AZ (US); Charles T. Hennemeyer, Tucson, AZ (US)

(21) Appl. No.: 17/792,684

(22) PCT Filed: Jan. 12, 2021
(86) PCT No.: PCT/US21/13097

§ 371 Date: Jul. 13, 2022

Related U.S. Application Data
(60) Provisional application No. 62/960,444, filed on Jan. 13, 2020.

(51) Int. Cl. G01N 1/30; G01N 33/58; G06V 10/143; G06V 20/69

(52) U.S. Cl. CPC G01N 1/30; G01N 33/582; G06V 10/143; G06V 20/693; G06V 20/695

(57) ABSTRACT

Optical biopsy staining panels for in vivo or in situ fluorescent staining of optical tissue (or other appropriate tissue), e.g., for the purpose of a direct biopsy such as an optical biopsy. The stain panels may feature a combination of a nuclear stain and a cytoplasmic stain, as a means of functioning as an invivo or in situ hematoxylin and eosin (H&E) stain. Examples of said stains may include anthracyclines such as Daunomcibin, acriflavines like Proflavine, anthracenediones such as Mitoxantrone, phenothiazines like Methylene Blue, and tri- and tetra-heterocyclic dyes like Fluorescein, Phloxine B, Phenol Red, Rose Bengal, Congo Red, and Indigo Carmine.

### CROSS-REFERENCES TO RELATED APPLICATIONS

This application claims benefit of U.S. Provisional Application No. 62/960,444, filed Jan. 13, 2020, the specification(s) of which is/are incorporated herein in their entirety by reference.

### BACKGROUND OF THE INVENTION

### Field of the Invention

The present invention relates to dyes and stains, more particularly to dyes and stains that can be used in vivo or in situ, such as for optical biopsies.

### Background Art

A biopsy is where tissue is examined under a microscope in order to obtain a cellular- and molecular-level diagnosis. Traditional biopsies (e.g., a physical biopsy) feature obtaining a piece of a patient's tissue, processing the tissue, and subsequently examining the tissue under the microscope. Unlike traditional biopsies, an optical biopsy involves taking the microscope directly into the patient's tissue. The benefits of a direct optical biopsy may include helping to decrease the rate of false-negative or inconclusive physical biopsy results, obviate the need for a physical biopsy, and provide for faster diagnoses.

The traditional biopsy process requires the tissue to be stained with various dyes to enable visualization of the microscopic architecture or cell type. The most commonly used stain is a hematoxylin and eosin (H&E) stain. Other stains that are commonly used include Gram stain, Cresyl violet, silver nitrate, Gomori Trichrome, Wright's blood, Feulgen reaction, Masson's Trichrome, Toluidine blue, Giemsa, Prussian Blue, etc. However, such dyes are not FDA approved for in vivo use. Optical biopsies are currently limited to what can be seen with visible or near visible wavelengths, limiting its usefulness since hemoglobin dominates the visual spectrum and naturally-fluorescing molecules do not provide sensitive or specific information.

A spectrum of drugs individually is currently FDA-approved for optical/fluorescence imaging, such as indocyanine green, fluorescein, oftasceine, trypan blue, etc. However, no combination of FDA-approved drugs has been developed to provide an adequate optical biopsy stain, such as an H&E equivalent.

### BRIEF SUMMARY OF THE INVENTION

One of the unique and inventive technical features of the present invention is the combination of certain drugs or dyes at particular optimal concentrations to create a fluorescent optical biopsy stain panel, such as an H&E equivalent. For example, certain combinations of drugs or dyes allow for emission of two (or more) different wavelengths (colors) upon excitation with one wavelength. Thus, the stain panels of the present invention may allow for multiplexing. None of the presently known prior references or work has the unique inventive technical feature of the present invention.

Furthermore, the prior references teach away from the present invention. For example, it is counterintuitive to use multiple dyes in an optical biopsy stain panel because there is often an overlap of the fluorescent emission signals of the different dyes. However, it was discovered that certain combinations of dyes at specific ratios of concentrations are distinct enough to be used in an optical biopsy stain panel. For example, FIGS. 8-15 show the emission spectra of different combinations of dyes at different concentrations. In the cases where there is a mixture of the dyes, there are distinct emission peaks for each dye.

In some aspects, the present invention features optical biopsy stain panels. In certain embodiments, the stain panels comprise two or more agents, wherein either (or all) of the agents fluorescently stains: nucleic acids; cytoplasm; cell or subcellular membranes; subcellular organelles; extracellular matrix components; or microbes.

In certain embodiments, one or more of the agents are clinical drugs. In certain embodiments, one or more of the agents are therapeutic drugs. In certain embodiments, one or more of the agents are supplements or food additives generally recognized as safe.

In certain embodiments, the agents are existing clinically-used agents. In some embodiments, the agents can be fluorescent nucleic acid stains selected from a group of nucleic acid-binding agents, such as, for example, aminoacridines, anthracyclines, anthracenediones, or phenothiazines. In certain embodiments, the aminoacridines are 9-aminmoacridine or proflavine, or others. In certain embodiments, the anthracyclines are Daunorubicin, Doxorubicin, Epirubicin, Idarubicin, Nogalamycin, or Valrubicin, or others. In certain embodiments, the anthracenediones are mitoxantrone or pixantrone, or others. In certain embodiments, the phenothiazine is methylene blue, or others.

In some other embodiments, the agents can be fluorescent cytoplasmic stains selected from a group of protein binding agents, such as heterotetracyclic and heteropentacyclic compounds, xanthylium dyes, azo dyes, indoles, or phthalein dyes. In certain embodiments, the heterotetracyclic and heteropentacyclic compounds are fluorescein or Rose Bengal, or others. In certain embodiments, the xanthylium dye is phloxine B, or others. In certain embodiments, the azo dye is Congo Red, or others. In certain embodiments, the indole is Indigo Carmine, or others. In certain embodiments, the phthalein dye is phenol red, or others.

In certain embodiments, the stains are existing clinically-used agents and fluorescent cell or subcellular membrane staining components, including drugs from Table 3 below. In certain embodiments, the stains are existing clinically-used agents and fluorescent organelle staining components, including drugs from Tables 4 and 5 below. In certain embodiments, the stains are existing clinically-used agents that stain extracellular matrix components. In certain embodiments, the stains are existing clinically-used agents that stain microbes.

In certain embodiments, the fluorescent nucleic acid stain is Daunorucibin and the fluorescent cytoplasmic stain is Fluorescein. In certain embodiments, the fluorescent nucleic acid stain is Methylene Blue and the fluorescent cytoplasmic stain is Fluorescein. In certain embodiments, the fluorescent nucleic acid stain is Methylene Blue and the fluorescent cytoplasmic stain is Phenol Red. In certain embodiments, the fluorescent nucleic acid stain is Methylene Blue and the fluorescent cytoplasmic stain is Phloxine B. In certain embodiments, the fluorescent nucleic acid stain is Methylene Blue and the fluorescent cytoplasmic stain is Rose Bengal. In certain embodiments, the fluorescent nucleic acid stain is Mitoxantrone and the fluorescent cytoplasmic stain is Fluorescein. In certain embodiments, the fluorescent nucleic acid stain is Mitoxantrone and the fluorescent cytoplasmic stain is Phenol Red. In certain embodiments, the fluorescent nucleic acid stain is Mitoxantrone and the fluorescent cytoplasmic stain is Phloxine B. In certain embodiments, the fluorescent nucleic acid stain is Mitoxantrone and the fluorescent cytoplasmic stain is Rose Bengal. In certain embodiments, the fluorescent nucleic acid stain is Proflavine and the fluorescent cytoplasmic stain is Rose Bengal.

In some embodiments, the present invention also features an apparatus where the illumination of an optical biopsy stain panel according to the present invention is about a single wavelength. In certain embodiments, the observation occurs through color filtering means or amplification or conversion of the resultant fluorescence into observable color range.

In other embodiments, the present invention features methods where fluorescence from an optical biopsy stain according to the present invention is observed even indirectly with the human eye.

In some embodiments, the present invention also features optical biopsy stain panels, where the panels comprise two or more agents. In one embodiment of the optical biopsy stain panels, one of the agents is a fluorescent nuclear stain. In another embodiment of the optical biopsy stain panels, one of the agents is a fluorescent cytoplasmic stain. In yet another embodiment of the optical biopsy stain panels, one agent is a fluorescent nuclear stain and another agent is a fluorescent cytoplasmic stain.

Note that while endoscopy, colonoscopy, bronchoscopy, arthroscopy, and other similar procedures may feature applying a microscope directly to tissue, the anatomy can be seen without the aid of fluorescence stains. For example, the procedures may rely on autofluorescence of tissue, or in some cases, the procedures utilize visual dyes for viewing in white lights. Without wishing to limit the present invention to any theory or mechanism, optical biopsies cannot rely on low-sensitivity autofluorescence of tissue, and the visual spectrum is often dominated by hemoglobin, limiting white light or non-fluorescence usefulness.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows proflavine, a topical antiseptic and wound irrigant, after being topically applied to a tissue sample (50 μl at 20 μM concentration) and imaged. Image field is 635.5 μm tall×wide. Inset is the structure of Proflavine.

FIG. 2A shows bovine lung tissue stained with Fluorescein (500 nM) and imaged using confocal microscopy excited with 488 nm wavelengths at various gains. Image field is 635.5 μm tall×wide.

FIG. 2B shows bovine lung tissue stained with Phenol Red (100 μM) and imaged using confocal microscopy excited with 488 nm wavelengths at various gains. Image field is 635.5 μm tall×wide.

FIG. 2C shows bovine lung tissue stained with Phloxine B (10 μM) and imaged using confocal microscopy excited with 488 nm wavelengths at various gains. Image field is 635.5 μm tall×wide.

FIG. 2D shows bovine lung tissue stained with Rose Bengal (100 μM) and imaged using confocal microscopy excited with 488 nm wavelengths at various gains. Image field is 635.5 μm tall×wide.

FIG. 2E shows the inset from FIG. 2A, which shows the fluorescence signal from fibers and variable cellular uptake.

FIG. 2F shows the inset from FIG. 2B, which shows the fluorescence signal from fibers and variable cellular uptake.

FIG. 2G shows the inset from FIG. 2C, which shows the fluorescence signal from fibers and variable cellular uptake.

FIG. 2H shows the inset from FIG. 2D, which shows the fluorescence signal from fibers and variable cellular uptake.

FIG. 2I shows an optimized autofluorescence signal, demonstrating prominent fibers but relative lack of cellular signal. Image field is 635.5 μm tall×wide.

FIG. 3 shows Daunorubicin and Fluorescein as DNA and protein/cytoplasm dyes, respectively. The combination of 25 μM Daunorubicin and 250 nM Fluorescein was added to bovine lung tissue samples and imaged using confocal microscopy with 488 nm excitation and 10 μm thick optical focal plane, with imaged fields being 635.5 μm². Inset shows emission spectra of the combination at 488 nm excitation with the microscope channels indicated along the abscissa.

FIG. 4A shows Methylene Blue as a DNA dye combined with an off-label protein/cytoplasm dye (50 μM Methylene Blue with 250 nM Fluorescein). Imaging was with confocal microscopy, with a bovine lung sample thickness of 10 μm, and an image field of 635.5 μm². Inset shows fluorescence emission spectra of the combination in similar concentration ratios also at 488 nm excitation, with the microscope detector channels along the abscissa.

FIG. 4B shows Methylene Blue as a DNA dye combined with an off-label protein/cytoplasm dye (100 μM Methylene Blue with 100 μM Phenol Red). Imaging was with confocal microscopy, with a bovine lung sample thickness of 10 μm, and an image field of 635.5 μm². Inset shows fluorescence emission spectra of the combination in similar concentration ratios also at 488 nm excitation, with the microscope detector channels along the abscissa.

FIG. 4C shows Methylene Blue as a DNA dye combined with an off-label protein/cytoplasm dye (100 μM Methylene Blue with 10 μM Phloxine B). Imaging was with confocal microscopy, with a bovine lung sample thickness of 10 μm, and an image field of 635.5 μm². Inset shows fluorescence emission spectra of the combination in similar concentration ratios also at 488 nm excitation, with the microscope detector channels along the abscissa.

FIG. 4D shows Methylene Blue as a DNA dye combined with an off-label protein/cytoplasm dye (100 μM Methylene Blue with 100 μM Rose Bengal). Imaging was with confocal microscopy, with a bovine lung sample thickness of 10 μm, and an image field of 635.5 μm². Inset shows fluorescence emission spectra of the combination in similar concentration ratios also at 488 nm excitation, with the microscope detector channels along the abscissa.

FIG. 5A shows Mitoxantrone as a DNA dye combined with a protein/cytoplasm dye (Mitoxantrone 50 μM and Fluorescein 250 nM). Imaging was with confocal microscopy settings at 488 nm excitation after topical application to the bovine lung specimen. Image field is 635.5 μm² at 10 μm thickness. Inset shows fluorescence emission spectra of the combination in similar ratios also at 488 nm excitation.

FIG. 5B shows Mitoxantrone as a DNA dye combined with a protein/cytoplasm dye (Mitoxantrone at 100 μM and Phenol Red 100 μM). Imaging was with confocal microscopy settings at 488 nm excitation after topical application to the bovine lung specimen. Image field is 635.5 μm² at 10 μm thickness. Inset shows fluorescence emission spectra of the combination in similar ratios also at 488 nm excitation.

FIG. 5C shows Mitoxantrone as a DNA dye combined with a protein/cytoplasm dye (Mitoxantrone 50 μM and Phloxine B 50 μM). Imaging was with confocal microscopy settings at 488 nm excitation after topical application to the bovine lung specimen. Image field is 635.5 μm² at 10 μm thickness. Inset shows fluorescence emission spectra of the combination in similar ratios also at 488 nm excitation.

FIG. 5D shows Mitoxantrone as a DNA dye combined with a protein/cytoplasm dye (Mitoxantrone 100 μM and Rose Bengal 100 μM). Imaging was with confocal microscopy settings at 488 nm excitation after topical application to the bovine lung specimen. Image field is 635.5 μm² at 10 μm thickness. Inset shows fluorescence emission spectra of the combination in similar ratios also at 488 nm excitation.

FIG. 6A shows Proflavine as a DNA drug-dye with Rose Bengal as a protein/cytoplasm drug-dye (50 μl of 100 μM Proflavine and 100 μM Rose Bengal). The dyes were pipetted in situ upon bovine tissue ex vivo and examined under confocal microscopy with a 488 nm laser with 10 μm optical thickness and imaged fields being 635.5 μm². Inset shows the fluorescence emission spectrum of the combination at 488 nm excitation.

FIG. 6B shows bovine lung tissue fixed in 10% formalin, paraffin embedded, cut to 5 μm sections and stained with Hematoxylin and Eosin per routine protocol, then imaged at 20×.

FIG. 6C shows an inversion of the colors of FIG. 6B, providing a black background. Image field is 1400×1052 μm.

FIG. 7 shows human breast cancer metastasized to the lung and stained with Rose Bengal and Proflavine to outline the proteins and nucleus, respectively, at concentrations similar to that in FIG. 6A. Image field is 625 μm².

FIG. 8A shows emission spectra of Mitoxantrone as a Hematoxylin-alternative candidate at 10 μM, with Phloxine B at 0.1 μM as an Eosin-alternative candidate drug-dye, excited at 375 nm.

FIG. 8B shows emission spectra of Mitoxantrone as a Hematoxylin-alternative candidate at 10 μM, with Phloxine B at 0.1 μM as an Eosin-alternative candidate drug-dye, excited at 405 nm.

FIG. 8C shows emission spectra of Mitoxantrone as a Hematoxylin-alternative candidate at 10 μM, with Phloxine B at 0.1 μM as an Eosin-alternative candidate drug-dye, excited at 455 nm.

FIG. 8D shows emission spectra of Mitoxantrone as a Hematoxylin-alternative candidate at 10 μM, with Phloxine B at 0.1 μM as an Eosin-alternative candidate drug-dye, excited at 488 nm.

FIG. 8E and FIG. 8F show concentration-dependent fluorescence of Mitoxantrone but not Phloxine B results in various concentration ratios of the two resulting in similar fluorescence intensity peaks, all excited at 455 nm. (Of note, smoothing after normalizing to peak intensity resulted in peaks not reaching exactly 1.0).

FIG. 9 shows an example of fluorescence incompatibility. At 488 nm excitation, increasing concentrations of Phloxine B (a proposed Eosin-like dye) resulted in progressive decrease of fluorescence emission from the Daunorubicin (a proposed Hematoxylin-like dye) fluorescence peak around 590 nm. Additional incompatibility results from overlapping of the component peak emissions.

FIG. 10 shows borderline compatibility of nucleic acid and cytoplasmic dyes. Actual (not normalized) fluorescence intensity of 10 μM Pyrvinium Pamoate decreased in the presence of 1 μM Rose Bengal, but not enough to obscure the Pyrvinium Pamoate component of the combined spectrum at 488 nm excitation.

FIG. 11 shows fluorescence emission spectra compatibility in vitro of the possible protein drug-dye Fluorescein with nucleic acid drug-dye Daunorubicin. Daunorubicin (1 μM), Fluorescein (1 nM) and the combination of the two were excited at 375 nm, with resultant fluorescence emission.

FIG. 12A shows fluorescence emission spectra compatibility in vitro of Methylene Blue as a DNA drug-dye with Fluorescein as a protein/cytoplasmic dye at 455 nm excitation.

FIG. 12B shows fluorescence emission spectra compatibility in vitro of Methylene Blue as a DNA drug-dye with 300 μM Phenol Red at pH 5 and 488 nm excitation.

FIG. 12C shows fluorescence emission spectra compatibility in vitro of Methylene Blue as a DNA drug-dye with Phloxine B at 2 μM at 520 nm excitation.

FIG. 12D shows fluorescence emission spectra compatibility in vitro of Methylene Blue as a DNA drug-dye with 1 μM Rose Bengal at 488 nm excitation.

FIG. 13A shows fluorescence emission spectra compatibility in vitro of the cytoplasmic drug-dye Fluorescein at 0.5 nM with nucleic acid drug-dye Mitoxantrone at 10 μM. The combination was excited at 375 nm, with the resultant emission spectrum shown.

FIG. 13B shows fluorescence emission spectra compatibility in vitro of the cytoplasmic drug-dye Phloxine B at 10 μM with nucleic acid drug-dye Mitoxantrone at 10 μM. The combination was excited at 405 nm, and the resultant spectrum displayed.

FIG. 13C shows fluorescence emission spectra compatibility in vitro of the cytoplasmic drug-dye Phenol Red at 100 μM with nucleic acid drug-dye Mitoxantrone at 10 μM. The combination of 10 μM Mitoxantrone and 10 μM Phenol Red at pH 5 was excited at 455 nm; the resultant spectrum is shown.

FIG. 13D shows fluorescence emission spectra compatibility in vitro of the cytoplasmic drug-dye Rose Bengal at 1 μM with nucleic acid drug-dye Mitoxantrone at 10 μM. The combination was excited at 455 nm and the resultant spectrum displayed.

FIG. 14 shows fluorescence emission spectra compatibility in vitro of Rose Bengal as a cytoplasmic drug-dye candidate with the nucleic acid drug-dye Proflavine. With 405 nm excitation, Proflavine at 0.25 μM, Rose Bengal at 10 μM, and the combination result in roughly the same order of magnitude peaks resulting from their respective fluorescence.

FIG. 15A shows fluorescence emission spectra compatibility in vitro of the cytoplasm-dye Fluorescein at 5 nM with nucleic acid drug-dye Pyrvinium Pamoate at 10 μM. The combination emission spectrum is shown with 455 nm excitation.

FIG. 15B shows fluorescence emission spectra compatibility in vitro of the cytoplasm-dye Phloxine B at 100 nM with nucleic acid drug-dye Pyrvinium Pamoate at 10 μM. The combination emission spectrum is shown with 455 nm excitation.

FIG. 15C shows fluorescence emission spectra compatibility in vitro of the cytoplasm-dye Phenol Red at 100 μM with nucleic acid drug-dye Pyrvinium Pamoate at 10 μM. The combination emission spectrum is shown with 488 nm excitation at approximately pH 5.

FIG. 15D shows fluorescence emission spectra compatibility in vitro of the cytoplasm-dye Rose Bengal at 1 μM with nucleic acid drug-dye Pyrvinium Pamoate at 10 μM. The combination emission spectrum is shown with 488 nm excitation.

### DETAILED DESCRIPTION OF THE INVENTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references.

All publications, patent applications, patents, and other references mentioned herein are incorporated by reference in their entirety for all purposes. In case of conflict, the present specification, including explanations of terms, will control.

The present invention features optical biopsy staining panels for in vivo or in situ staining of tissue, e.g., for the purpose of a direct biopsy such as an optical biopsy, or in conjunction with a physical biopsy followed by optical biopsy of the harvested tissue. While the compounds used in the optical biopsy staining panels may be FDA-approved chromogenic or fluorescent drugs, none have been used for fluorescent optical staining. Further, it was surprisingly discovered that combinations of said drugs at specific concentrations could be used as fluorescent stain panels.

Table 1 below shows examples of FDA-approved chromogenic/fluorescent drugs and their current uses

| Chromogenic/fluorescent drug | General in vivo Use/Treatment |
|---|---|
| Brilliant Blue G (Acid Blue, Coomassie | Ophthalmic contrast |
| Brilliant Blue) | |
| Brilliant Green | Topical antiseptic additive |
| Congo Red | Chromoendoscopy contrast agent |
| Crystal/Gentian Violet | Topical antiseptic additive, ophthalmic contrast |
| Fluorescein | Ophthalmic contrast, fluorescence angiography |
| Indigo Carmine | Neurosurgical, Gastrointestinal and Urological contrast |
| Indocyanine Green | Fluorescence angiography, lymphangiography |
| Isosulfan Blue | Lymphangiography |
| Lugol's Iodine Solution | Topical antiseptic, Stain for Schiller's Test |
| Methylene Blue | Antidote for methemoglobinemia, ophthalmic contrast |
| Oftasceine | Ophthalmic contrast |
| Patent Blue (Sulfan Blue) | Lymphangiography |
| Phenol Red | Chromoendoscopy |
| Phloxine B | Dental disclosing agent |
| Porphyrinoids/Porphyrin precursors | Neurosurgical and Urological surgery contrast |
| (Hexview/Cysview, 5-ALA) | |
| Prussian Blue | Antidote for cesium and thallium poisoning |
| Rose Bengal Free Acid | Ophthalmic contrast |
| Toluidine Blue | Visual diagnostic aid on mucosal lesions |
| Trypan Blue | Ophthalmic contrast |

Table 2 below shows non-limiting examples of optical biopsy stain panels according to the present invention. The biopsy stain panels may, for example, be equivalent to a traditional H&E stain. For example, in certain embodiments, the compound in the “Dye 1” category represents a nuclear/DNA stain and the compounds in the “Dye 2” category represent a protein/cytoplasm stain.

| Example | Dye 1 | Dye 2 |
|---|---|---|
| 1 | Daunorucibin | Fluorescein |
| 2 | Methylene Blue | Fluorescein |
| 3 | Proflavine | Fluorescein |
| 4 | Acridine Orange | Fluorescein |
| 5 | Mitoxantrone | Fluorescein |
| 6 | Pyrvinium Pamoate | Fluorescein |
| 7 | Daunorucibin | Phloxine B |
| 8 | Methylene Blue | Phloxine B |
| 9 | Proflavine | Phloxine B |
| 10 | Acridine Orange | Phloxine B |
| 11 | Mitoxantrone | Phloxine B |
| 12 | Pyrvinium Pamoate | Phloxine B |
| 13 | Daunorucibin | Phenol Red |
| 14 | Methylene Blue | Phenol Red |
| 15 | Proflavine | Phenol Red |
| 16 | Acridine Orange | Phenol Red |
| 17 | Mitoxantrone | Phenol Red |
| 18 | Pyrvinium Pamoate | Phenol Red |
| 19 | Daunorucibin | Rose Bengal |
| 20 | Methylene Blue | Rose Bengal |
| 21 | Proflavine | Rose Bengal |
| 22 | Acridine Orange | Rose Bengal |
| 23 | Mitoxantrone | Rose Bengal |
| 24 | Pyrvinium Pamoate | Rose Bengal |
| 25 | Daunorucibin | Congo Red |
| 26 | Methylene Blue | Congo Red |
| 27 | Proflavine | Congo Red |
| 28 | Acridine Orange | Congo Red |
| 29 | Mitoxantrone | Congo Red |
| 30 | Pyrvinium Pamoate | Congo Red |
| 31 | Daunorucibin | Indigo Carmine |
| 32 | Methylene Blue | Indigo Carmine |
| 33 | Proflavine | Indigo Carmine |
| 34 | Acridine Orange | Indigo Carmine |
| 35 | Mitoxantrone | Indigo Carmine |
| 36 | Pyrvinium Pamoate | Indigo Carmine |

Table 3 below shows non-limiting examples of clinically-used agents also working as lipid membrane stains based on similarity to membrane bilayer research dyes.

| | Research membrane dyes | | | | Specific Drugs with similarity to |
|---|---|---|---|---|---|
| | | | | Heptamethine | research dyes (>0.45 |
| Drug Class | Cy5 | CY7 | DiO/I | cyanine dye-1 | DrugBank.ca similarity score) |
| (Proto)Porphyrins | X | X | X | X | Hydroxocobalamin, |
| | | | | | Cyanocobalamin, Verteporfin |
| Ace inhibitor | X | | | | Benazepril |
| Adrenergics/ | X | | X | X | Ergotamine, Silodosin, Yohimbine |
| dopaminergics | | | | | |
| Antihyperglycemic | X | | | | Repaglinide |
| Antineoplastic | X | X | X | X | Vindesine, Bleomycin, Vinblastine |
| Bacitracin | | X | | | Bacitracin A |
| Carbapenems | X | X | X | X | Ertapenem |
| Cefalosporins | | | X | X | Cefpirome, Cefpiramide |
| Cyclooxygenase | | X | | | Meloxicam, Piroxicam |
| inhibitor | | | | | |
| Indigotindisulfonic | | | X | | Indigotindisulonic Acid |
| acid | | | | | |
| Kinase inhibitors | X | X | X | | Nintedanib |
| Na+ channel blockers | | X | X | | Ajmaline |
| Non-dihydropyridine | | X | | | Diltiazem |
| calcium channel | | | | | |
| blockers | | | | | |
| Opioids | | X | | | Codeine, Morphine |
| Phosphodiesterase | | X | | | Vardenafil, Sildenafil |
| inhibitor | | | | | |
| Rifamycins | X | X | X | X | Rifabutin, Rifapentine, Rifaximin |
| Ristocetins | | X | X | | Vancomycin, Oritovancin |
| Tetracyclines | X | X | X | X | Tigecycline |

Table 4 below shows non-limiting examples of clinically-used agents with localization to mitochondria as the prototypical subcellular organelle.

| | Mitochondrial Dyes | | | |
|---|---|---|---|---|
| | Mitotracker | Mitotracker | Mitotracker | Mitotracker |
| Drugs with similarity score >0.45 | Orange | Deep Red | Red | Green |

| | | |
|---|---|---|
| Antimitotic: Vinorelbine, Vinblasine, | X | X |
| Vincristine, Vinflunine, Bleomycin | | |
| Opioids: Pholcodine, | X | |
| Benzhydrocodone, Oxycodone, | | |
| Morphine, Codeine, Hydrocodone, | | |
| Naltrexone, Methylnaltrexone, | | |
| Naloxone, Oxymorphone | | |
| Restocins: Vancomycin, Telavancin | X | |
| Rifamycins: Rifaximin, Rifapentine | X | |
| Porphyrins: Cyanocobalamin, | X | X |
| Cobamamide | | |
| Atypical Antihistamine: Tritoqualine | X | |
| Cephalosporins: Ceftolozane, | | X |
| Ceftaroline Fosamil, Ceftriaxone, | | |
| Cefpirome | | |
| Anti-Insomnia: Suvorexant | | X |
| Anti-inflammatory: Benoxaprofen | | X |
| Antineoplastic: Trabectedin | | X |

Table 5 below shows non-limiting examples of clinically-used agents with localization to lysosomes and related organelles similar to research dyes.

| | Lysosome Dyes | | | | | | |
|---|---|---|---|---|---|---|---|
| | Lysotracker | Lysotracker | Lysotracker | Lysotracker | Lysosensor | Lysosensor | Lysosensor |
| Drugs with similarity score >0.5 | Red | Yellow | Green | Blue | Blue | Yellow | Green |
| Antifungal: Butenafine, Naftifine, | | | | X | X | | |
| Terbinafine | | | | | | | |
| Hematopoietic Stem Cell Mobilizer: | | | | X | X | | |
| Plerixafor | | | | | | | |
| H1 Antagonist: Cyclizine, Hydroxyzine | | | | X | X | | |
| Maoi: Pargyline | | | | X | | | |
| Class 1 Antiarrythmics: Procainamide | | | | X | | | |
| Porphyrins: Cobamamide, | X | | | | | | |
| Methylcobalamin, Hydroxocobalamin, | | | | | | | |
| Cyanocobalamin | | | | | | | |
| Antimitotics: Vinorelbine, Vinflunine, | X | | | | | | |
| Vindesine, Vinblastine, Bleomycin | | | | | | | |
| Antiviral: Voxilaprevir, Grazoprevir, | X | | | | | | |
| Glecaprevir, Elbasvir, Simeprevir | | | | | | | |
| Topoisomerase Inhibitor: Irinotecan | X | | | | | | |
| Cephalosporins: Cefpirome, Cefpiramide, | X | | | | | | |
| Ceftriaxone, Ceftazidime, Cefixime, | | | | | | | |
| Cefotaxime, Cefmenoxime | | | | | | | |
| Dopaminergic/Adrenergic: Bromocriptine, | X | | | | | | |
| Ergotamine, Dihydroergotamine, | | | | | | | |
| Reserpine, Deserpine | | | | | | | |
| Rifamycins: Rifapentine, | X | | | | | | |
| Rifabutin, Rifampicin | | | | | | | |
| Opioid: Naldemedine | X | | | | | | |
| Non-Steroidal Antiinflammatory: | | | | | | X | |
| Oxaprozin | | | | | | | |
| Broad-Spectrum Antimicrobial: | | | | | X | | |
| Benzethonium | | | | | | | |

The present invention is not limited to the aforementioned examples of optical biopsy stain panels.

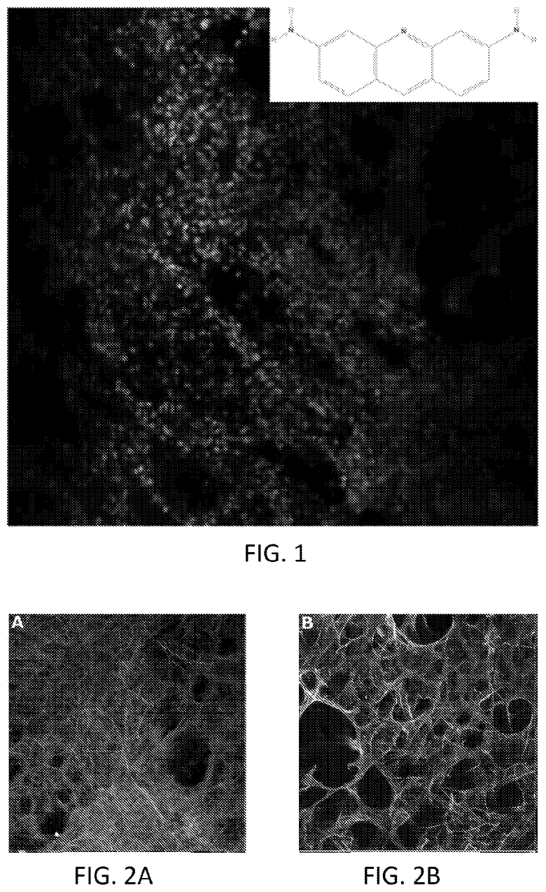

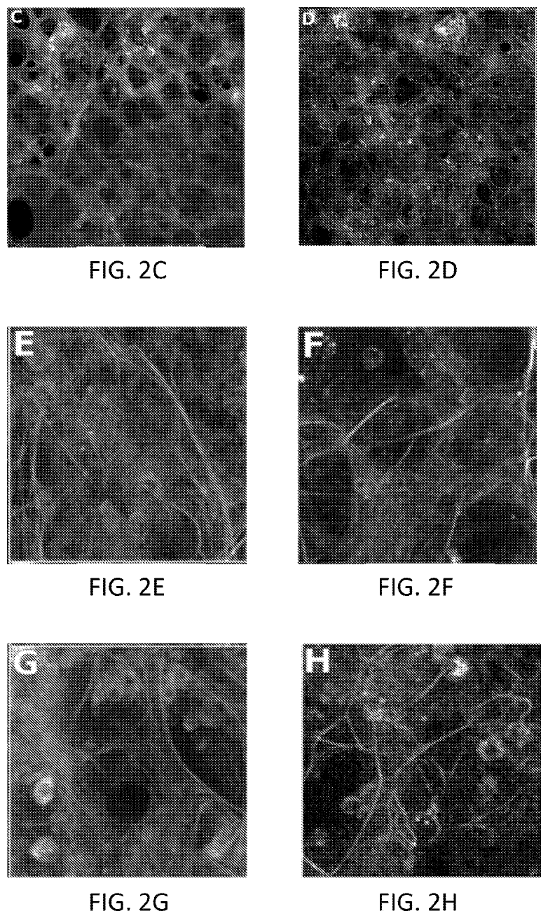

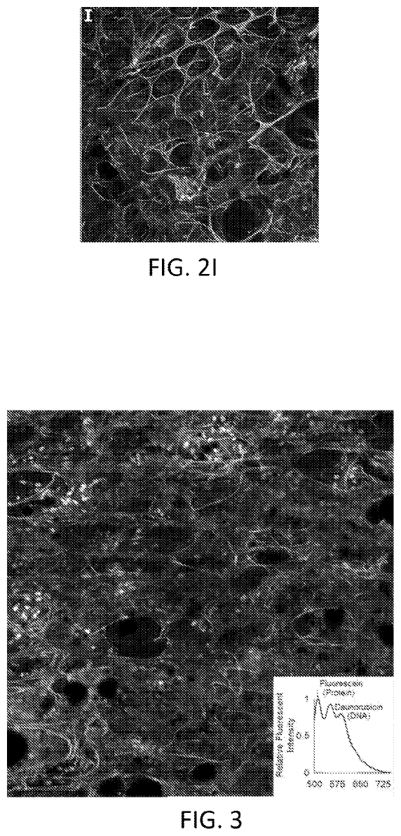

Referring to FIG. 1, 50 μl at 20 μM of Proflavine was topically applied directly to ex vivo bovine lung tissue in situ and imaged immediately with confocal microscopy. The drug-dye Proflavin showed staining like those typical of a fluorescent DNA dye. Likewise, Fluorescein, Phenol Red, Phloxine B, and Rose Bengal were each added to ex vivo bovine lung tissue and imaged under confocal microscopy (see FIGS. 2A-2I). Each drug-dye resulted in variable staining of the proteinaceous connective tissue and cellular cytoplasm, granules or vesicles. Qualitatively at these concentrations, Fluorescein, Phloxine B and Rose Bengal drug-dyes appeared to localize to cell cytoplasm in addition to the connective tissue fibers, which fibers also demonstrated autofluorescence. Fluorescein was the only protein/cytoplasm dye without excessive overlap with the resultant fluorescence signal from Daunorubicin as a DNA-dye. This combination was then added topically to bovine lung tissue and imaged using confocal microscopy (FIG. 3).

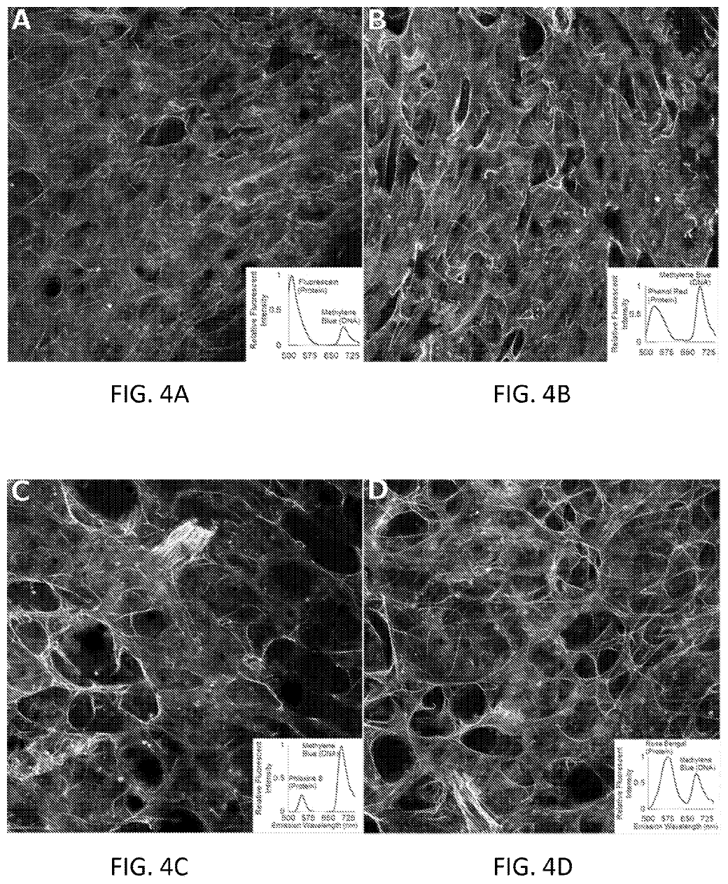

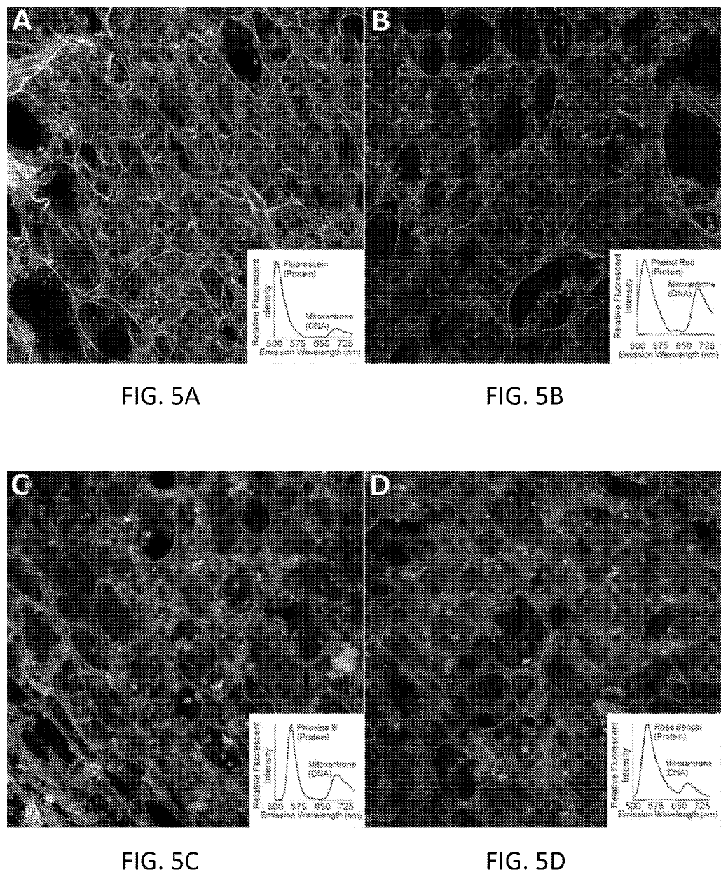

Methylene Blue was tested as a fluorescent DNA drug-dye in combination with several protein/cytoplasm dyes. These combinations were applied topically to bovine lung samples and imaged using confocal microscopy at 488nm excitation (see FIGS. 4A-4D). Also, mitoxantrone was tested as a DNA dye in combination with several protein/cytoplasm drug-dyes. Again, the combinations were topically applied to bovine lung and examined with confocal microscopy at 488 nm excitation (see FIGS. 5A-5D).

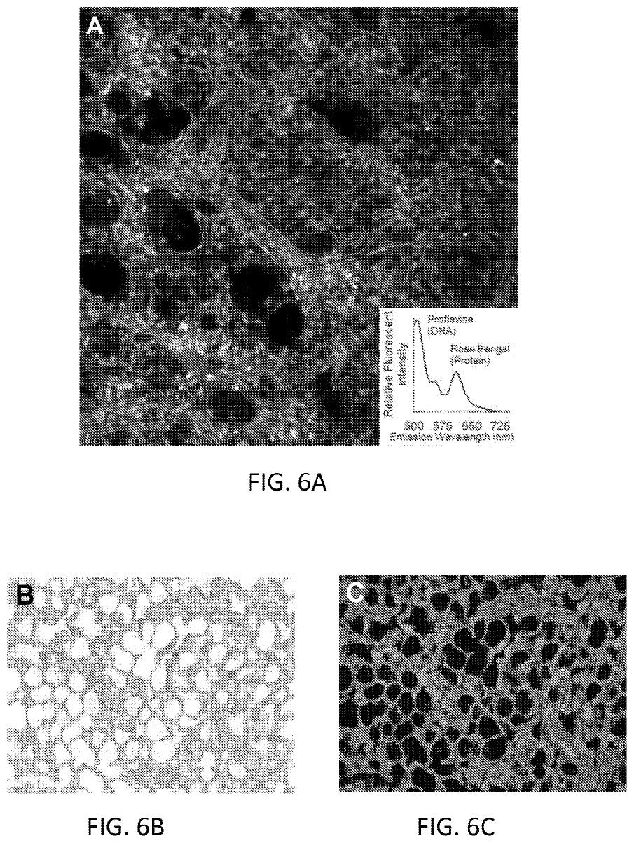

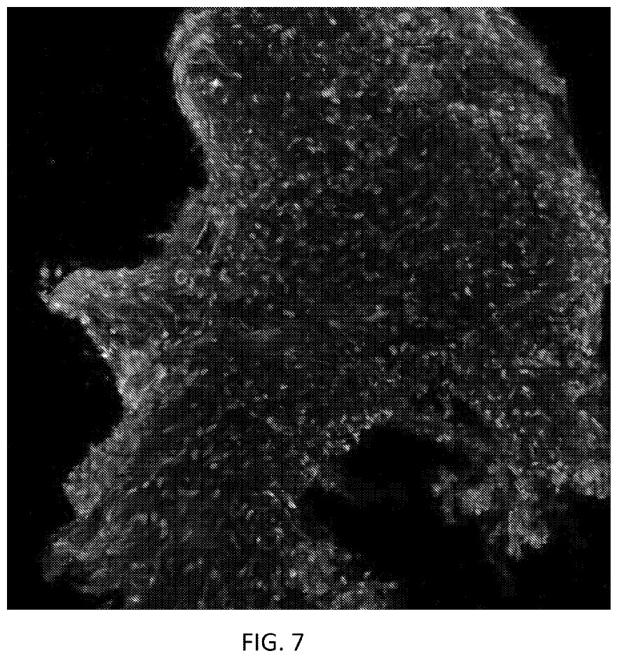

Lastly, a combination of Proflavine and Rose Bengal was tested on bovine lung samples and evaluated with confocal microscopy (see FIG. 6A). Since H&E is the gold-standard to which these new optical stain panels are compared, bovine lung tissue was fixed in formalin, embedded in paraffin and sectioned at 5 μm thickness and finally stained with Hematoxylin and Eosin per routine protocol (see FIG. 6B). To provide a more intuitive comparison in making the background black, the color was inverted (see FIG. 6C).

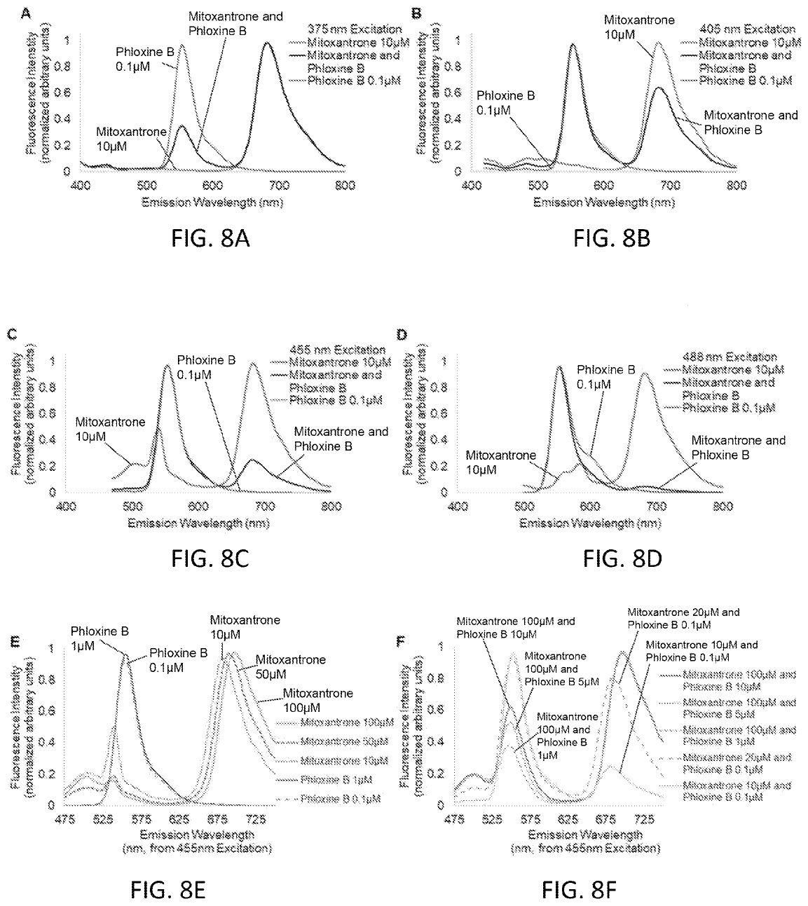

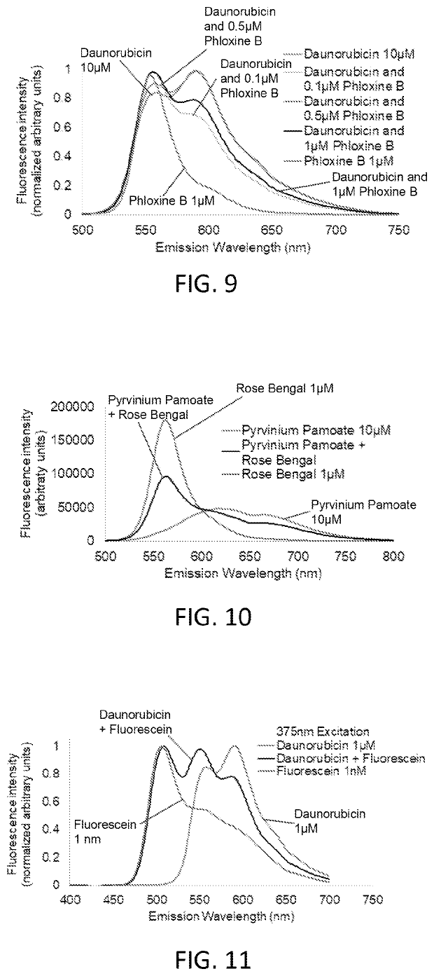

FIGS. 8A-8F show emission spectra of various drug dye candidates. FIGS. 8A-8D show Mitoxantrone as a Hematoxylin-alternative candidate at 10 μM, with Phloxine B at 0.1 μM as an Eosin-alternative candidate drug-dye, and a combination of the two excited at different wavelengths: (FIG. 8A) 375 nm, (FIG. 8B) 405 nm, (FIG. 80) 455 nm, and (FIG. 8D) 488 nm. FIGS. 8E-8F show concentration-dependent fluorescence of Mitoxantrone but not Phloxine B results in various concentration ratios of the two resulting in similar fluorescence intensity peaks, all excited at 455 nm. The molar ratios varied primarily based on excitation wavelength, although some dyes demonstrated concentration-dependent change in fluorescence which resulted in unexpected changes in the molar ratio required for similar peak emission intensity. Specifically, Mitoxantrone as a DNA dye mixed with Phloxine B as a non-specific protein dye at 100:1 molar ratio resulted in primarily Mitoxantrone from the resultant fluorescence emission signal at 375 nm excitation (FIG. 8A), but nearly all the fluorescence emission signal from Phloxine B at 488 nm excitation (FIG. 8D). Additionally, there was overlap of emission spectra (FIG. 9) and the combination of certain DNA and protein dyes decreased the emission intensity relative to the same concentration of the component dye alone (FIG. 9 and FIG. 10).

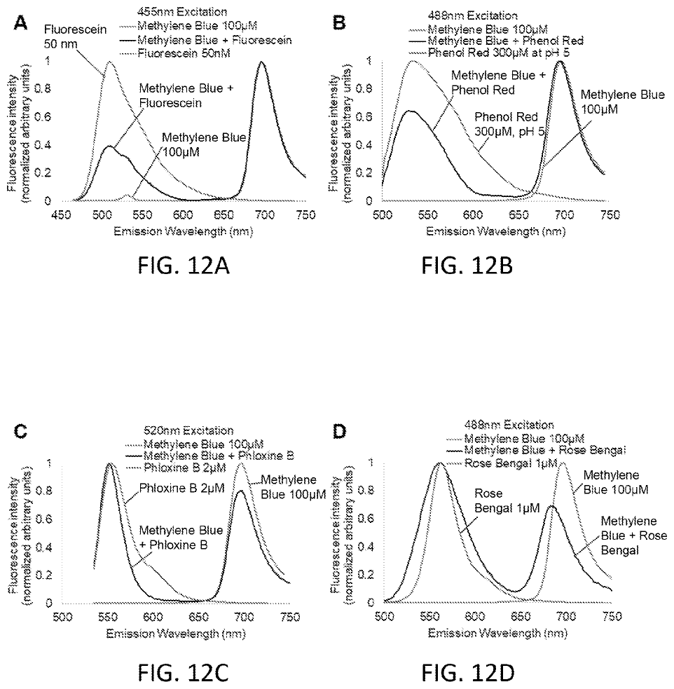

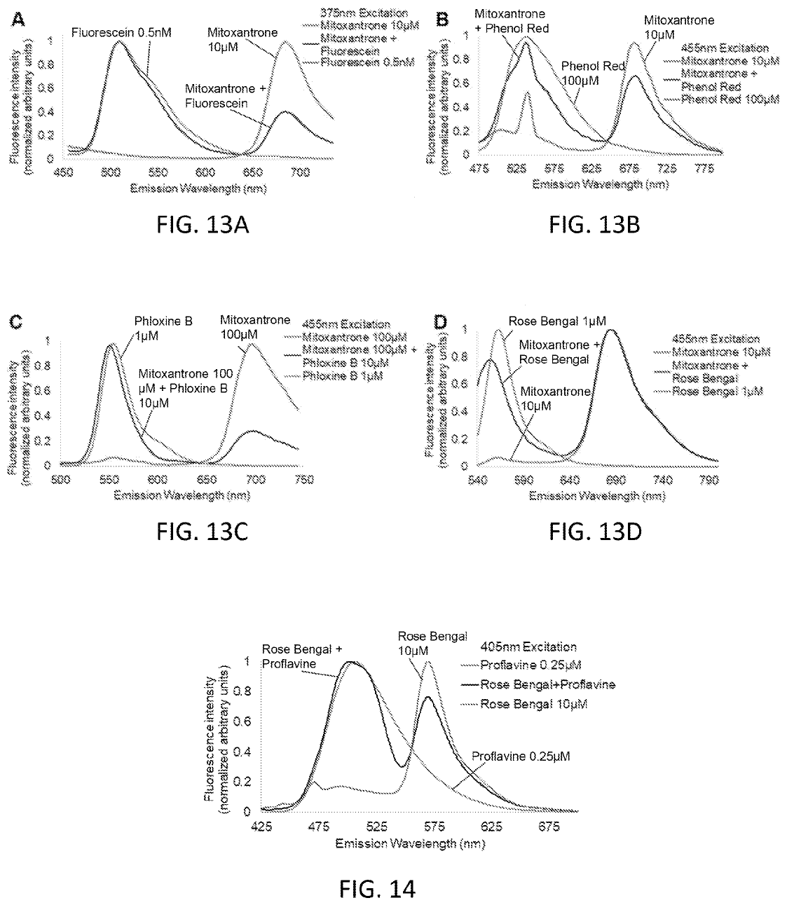

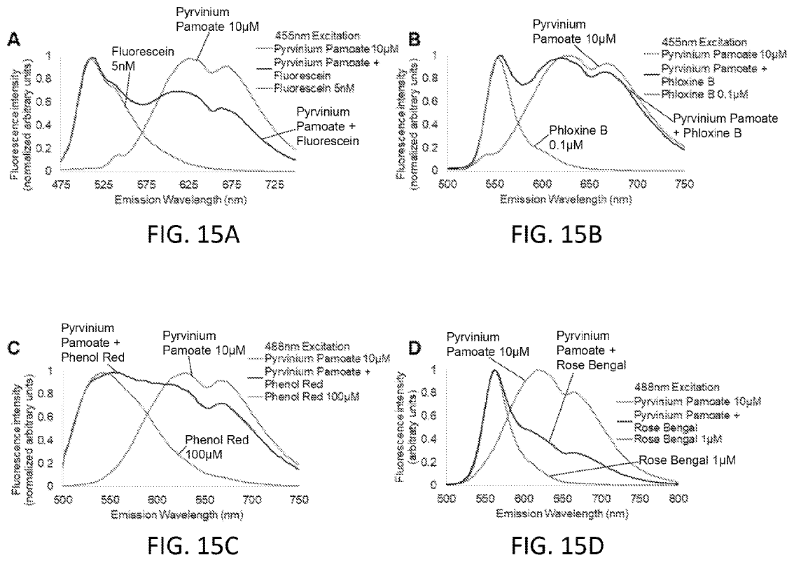

FIGS. 11-15 demonstrate individual and combined fluorescence emission spectra of particular DNA dyes and protein/cytoplasm dyes. Of note, Pyrvinium Pamoate was not further evaluated based on substantial overlap of the emission spectra of all the protein/cytoplasm dyes.

The present invention also features methods of use of the optical biopsy stain panels of the present invention. In certain embodiments, the methods feature injecting one or a combination of stains (e.g., in series, at the same time) into the target tissue, introducing the microscope (e.g., microendocsope) to the tissue, and viewing the stain. In certain embodiments, the methods herein provide for a provisional diagnosis and allow for determining whether or not to proceed with a physical biopsy.

The present invention also features kits comprising one or a combination of dyes disclosed herein, e.g., the combinations disclosed in Table 2. For example, in certain embodiments, the kit comprises Daunorucibin and Fluorescein; in certain embodiments, the kit comprises Proflavine and Rose Bengal; etc.

Table 6 below shows non-limiting examples of optical biopsy stain panels and the parts of the cell that are fluorescently labeled.

| | 9- | Profla- | Dauno- | Doxo- | | |
|---|---|---|---|---|---|---|
| | aminoacridine | vine | rubicin | rubicin | Epirubicin | Idarubicin |
| Fluorecein | Nucleic | Nucleic | Nucleic | Nucleic | Nucleic | Nucleic |
| | acids + | acids + | acids + | acids + | acids + | acids + |
| | cytoplasm | cytoplasm | cytoplasm | cytoplasm | cytoplasm | cytoplasm |
| Rose | Nucleic | Nucleic | Nucleic | Nucleic | Nucleic | Nucleic |
| Bengal | acids + | acids + | acids + | acids + | acids + | acids + |
| | cytoplasm | cytoplasm | cytoplasm | cytoplasm | cytoplasm | cytoplasm |
| Phloxine | Nucleic | Nucleic | Nucleic | Nucleic | Nucleic | Nucleic |
| B | acids + | acids + | acids + | acids + | acids + | acids + |
| | cytoplasm | cytoplasm | cytoplasm | cytoplasm | cytoplasm | cytoplasm |
| Congo | Nucleic | Nucleic | Nucleic | Nucleic | Nucleic | Nucleic |
| Red | acids + | acids + | acids + | acids + | acids + | acids + |
| | cytoplasm | cytoplasm | cytoplasm | cytoplasm | cytoplasm | cytoplasm |
| Indigo | Nucleic | Nucleic | Nucleic | Nucleic | Nucleic | Nucleic |
| Carmine | acids + | acids + | acids + | acids + | acids + | acids + |
| | cytoplasm | cytoplasm | cytoplasm | cytoplasm | cytoplasm | cytoplasm |
| Phenol | Nucleic | Nucleic | Nucleic | Nucleic | Nucleic | Nucleic |
| Red | acids + | acids + | acids + | acids + | acids + | acids + |
| | cytoplasm | cytoplasm | cytoplasm | cytoplasm | cytoplasm | cytoplasm |
| Hydroxycobalamin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Cyanocobalamin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Verteporfin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Benazepril | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Ergotamine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Siiodosin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Yohimbine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Repaglinide | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Vindesine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Bleomycin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Vinblastine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Bacitracin | Nucleic | Nucleic | Nucleic | Nucleic | Nucleic | Nucleic |
| A | acids + | acids + | acids + | acids + | acids + | acids + |
| | lipid | lipid | lipid | lipid | lipid | lipid |
| | membranes | membranes | membranes | membranes | membranes | membranes |
| Ertapenem | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Cefpirome | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Cefpiramide | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Meloxicam | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Piroxicam | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Indigotindisulonic | Nucleic | Nucleic | Nucleic | Nucleic | Nucleic | Nucleic |
| Acid | acids + | acids + | acids + | acids + | acids + | acids + |
| | lipid | lipid | lipid | lipid | lipid | lipid |
| | membranes | membranes | membranes | membranes | membranes | membranes |
| Nintedanib | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Ajmaline | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Diltiazem | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Codeine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| | or | or | or | or | or | or |
| | mitochondria | mitochondria | mitochondria | mitochondria | mitochondria | mitochondria |
| Morphine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| | or | or | or | or | or | or |
| | mitochondria | mitochondria | mitochondria | mitochondria | mitochondria | mitochondria |
| Vardenafil | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Slidenafil | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Rifabutin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Rifapentine | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| | or | or | or | or | or | or |
| | mitochondria | mitochondria | mitochondria | mitochondria | mitochondria | mitochondria |
| Rifaximin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| | or | or | or | or | or | or |
| | mitochondria | mitochondria | mitochondria | mitochondria | mitochondria | mitochondria |
| Vaneomycin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Oritovancin | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Tigecycline | Nucleic | Nucleic | Nucleic | Nucleic | Nucleic | Nucleic |
| | acids + | acids + | acids + | acids + | acids + | acids + |
| | lipid | lipid | lipid | lipid | lipid | lipid |
| | membranes | membranes | membranes | membranes | membranes | membranes |
| Vinorelbine | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Vinblastine | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Vincristine | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Vinflunine | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Bieomycin | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Pholcodine | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Benzhydrocodone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Oxycodone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Hydrocodone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Naltrexone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Methylnaltrexone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Naloxone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Oxymorphone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Telavancin | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Cyanocobalamin | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Cobamamide | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Tritoqualine | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Ceftoiozane | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Ceftaroline | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Fosamil | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Ceftriaxone | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Cefpirome | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| | or | or | or | or | or | or |
| | lysosome | lysosome | lysosome | lysosome | lysosome | lysosome |
| Suvorexant | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Benoxaprofen | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Trabectedin | Nucleic | Nucleic | Nucleic | Nucleic | Nucleic | Nucleic |
| | acids + | acids + | acids + | acids + | acids + | acids + |
| | mitochondria | mitochondria | mitochondria | mitochondria | mitochondria | mitochondria |
| Butenafine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Naftifine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Terbinafine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Plerixafor | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Pargyline | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Procainamide | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Methylcobalamin | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Hydroxo- | Nucleic | Nucleic | Nucleic | Nucleic | Nucleic | Nucleic |
| cobalamin | acids + | acids + | acids + | acids + | acids + | acids + |
| | lysosome | lysosome | lysosome | lysosome | lysosome | lysosome |
| Voxilaprevir | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Grazoprevir | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Glecaprevir | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Elbasvir | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Simeprevir | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Irinotecan | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Cefpiramide | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Ceftazidime | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Cefixime | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Cefotaxime | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Cefmenoxime | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Bromocriptine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Ergotamine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Dihydroer- | Nucleic | Nucleic | Nucleic | Nucleic | Nucleic | Nucleic |
| gotamine | acids + | acids + | acids + | acids + | acids + | acids + |
| | lysosome | lysosome | lysosome | lysosome | lysosome | lysosome |
| Reserpine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Deserpine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Rifabutin | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Naldemedine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Oxaprozin | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Benzethonium | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Rescinnamine | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Lymecycline | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Moxifloxacin | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Dactinomycin | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |
| Ertapenem | Nucleic | Nucleic | Nucleic | Nucleic | Nucleic | Nucleic |
| | acids + | acids + | acids + | acids + | acids + | acids + |
| | lysosome | lysosome | lysosome | lysosome | lysosome | lysosome |

| | | | Mitoxan- | | Methylene |
|---|---|---|---|---|---|
| | Nogalamycin | Valrubicin | trone | Pixantrone | Blue |
| Fluorecein | Nucleic | Nucleic | Nucleic | Nucleic | Nucleic |
| | acids + | acids + | acids + | acids + | acids + |
| | cytoplasm | cytoplasm | cytoplasm | cytoplasm | cytoplasm |
| Rose | Nucleic | Nucleic | Nucleic | Nucleic | Nucleic |
| Bengal | acids + | acids + | acids + | acids + | acids + |
| | cytoplasm | cytoplasm | cytoplasm | cytoplasm | cytoplasm |
| Phloxine | Nucleic | Nucleic | Nucleic | Nucleic | Nucleic |
| B | acids + | acids + | acids + | acids + | acids + |
| | cytoplasm | cytoplasm | cytoplasm | cytoplasm | cytoplasm |
| Congo | Nucleic | Nucleic | Nucleic | Nucleic | Nucleic |
| Red | acids + | acids + | acids + | acids + | acids + |
| | cytoplasm | cytoplasm | cytoplasm | cytoplasm | cytoplasm |
| Indigo | Nucleic | Nucleic | Nucleic | Nucleic | Nucleic |
| Carmine | acids + | acids + | acids + | acids + | acids + |
| | cytoplasm | cytoplasm | cytoplasm | cytoplasm | cytoplasm |
| Phenol | Nucleic | Nucleic | Nucleic | Nucleic | Nucleic |
| Red | acids + | acids + | acids + | acids + | acids + |
| | cytoplasm | cytoplasm | cytoplasm | cytoplasm | cytoplasm |
| Hydroxycobalamin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Cyanocobalamin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Verteporfin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Benazepril | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Ergotamine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Siiodosin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Yohimbine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Repaglinide | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Vindesine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Bleomycin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Vinblastine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Bacitracin | Nucleic | Nucleic | Nucleic | Nucleic | Nucleic |
| A | acids + | acids + | acids + | acids + | acids + |
| | lipid | lipid | lipid | lipid | lipid |
| | membranes | membranes | membranes | membranes | membranes |
| Ertapenem | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Cefpirome | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Cefpiramide | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Meloxicam | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Piroxicam | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Indigotindisulonic | Nucleic | Nucleic | Nucleic | Nucleic | Nucleic |
| Acid | acids + | acids + | acids + | acids + | acids + |
| | lipid | lipid | lipid | lipid | lipid |
| | membranes | membranes | membranes | membranes | membranes |
| Nintedanib | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Ajmaline | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Diltiazem | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Codeine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| | or | or | or | or | or |
| | mitochondria | mitochondria | mitochondria | mitochondria | mitochondria |
| Morphine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| | or | or | or | or | or |
| | mitochondria | mitochondria | mitochondria | mitochondria | mitochondria |
| Vardenafil | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Slidenafil | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Rifabutin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Rifapentine | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| | or | or | or | or | or |
| | mitochondria | mitochondria | mitochondria | mitochondria | mitochondria |
| Rifaximin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| | or | or | or | or | or |
| | mitochondria | mitochondria | mitochondria | mitochondria | mitochondria |
| Vaneomycin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Oritovancin | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Tigecycline | Nucleic | Nucleic | Nucleic | Nucleic | Nucleic |
| | acids + | acids + | acids + | acids + | acids + |
| | lipid | lipid | lipid | lipid | lipid |
| | membranes | membranes | membranes | membranes | membranes |
| Vinorelbine | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Vinblastine | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Vincristine | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Vinflunine | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Bieomycin | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Pholcodine | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Benzhydrocodone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Oxycodone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Hydrocodone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Naltrexone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Methylnaltrexone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Naloxone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Oxymorphone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Telavancin | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Cyanocobalamin | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Cobamamide | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Tritoqualine | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Ceftoiozane | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Ceftaroline | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Fosamil | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Ceftriaxone | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Cefpirome | Nucleic | Nucleic | Nucleic | Nucleic | Nucleic |
| | acids + | acids + | acids + | acids + | acids + |
| | mitochondria | mitochondria | mitochondria | mitochondria | mitochondria |
| | or | or | or | or | or |
| | lysosome | lysosome | lysosome | lysosome | lysosome |
| Suvorexant | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Benoxaprofen | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Trabectedin | Nucleic | Nucleic | Nucleic | Nucleic | Nucleic |
| | acids + | acids + | acids + | acids + | acids + |
| | mitochondria | mitochondria | mitochondria | mitochondria | mitochondria |
| Butenafine | Nucleic | Nucleic | Nucleic | Nucleic | Nucleic |
| | acids + | acids + | acids + | acids + | acids + |
| | lysosome | lysosome | lysosome | lysosome | lysosome |
| Naftifine | Nucleic | Nucleic | Nucleic | Nucleic | Nucleic |
| | acids + | acids + | acids + | acids + | acids + |
| | lysosome | lysosome | lysosome | lysosome | lysosome |
| Terbinafine | Nucleic | Nucleic | Nucleic | Nucleic | Nucleic |
| | acids + | acids + | acids + | acids + | acids + |
| | lysosome | lysosome | lysosome | lysosome | lysosome |
| Plerixafor | Nucleic | Nucleic | Nucleic | Nucleic | Nucleic |
| | acids + | acids + | acids + | acids + | acids + |
| | lysosome | lysosome | lysosome | lysosome | lysosome |
| Pargyline | Nucleic | Nucleic | Nucleic | Nucleic | Nucleic |
| | acids + | acids + | acids + | acids + | acids + |
| | lysosome | lysosome | lysosome | lysosome | lysosome |
| Procainamide | Nucleic | Nucleic | Nucleic | Nucleic | Nucleic |
| | acids + | acids + | acids + | acids + | acids + |
| | lysosome | lysosome | lysosome | lysosome | lysosome |
| Methylcobalamin | Nucleic | Nucleic | Nucleic | Nucleic | Nucleic |
| | acids + | acids + | acids + | acids + | acids + |
| | lysosome | lysosome | lysosome | lysosome | lysosome |
| Hydroxo- | Nucleic | Nucleic | Nucleic | Nucleic | Nucleic |
| cobalamin | acids + | acids + | acids + | acids + | acids + |
| | lysosome | lysosome | lysosome | lysosome | lysosome |
| Voxilaprevir | Nucleic | Nucleic | Nucleic | Nucleic | Nucleic |
| | acids + | acids + | acids + | acids + | acids + |
| | lysosome | lysosome | lysosome | lysosome | lysosome |
| Grazoprevir | Nucleic | Nucleic | Nucleic | Nucleic | Nucleic |
| | acids + | acids + | acids + | acids + | acids + |
| | lysosome | lysosome | lysosome | lysosome | lysosome |
| Glecaprevir | Nucleic | Nucleic | Nucleic | Nucleic | Nucleic |
| | acids + | acids + | acids + | acids + | acids + |
| | lysosome | lysosome | lysosome | lysosome | lysosome |
| Elbasvir | Nucleic | Nucleic | Nucleic | Nucleic | Nucleic |
| | acids + | acids + | acids + | acids + | acids + |
| | lysosome | lysosome | lysosome | lysosome | lysosome |
| Simeprevir | Nucleic | Nucleic | Nucleic | Nucleic | Nucleic |
| | acids + | acids + | acids + | acids + | acids + |
| | lysosome | lysosome | lysosome | lysosome | lysosome |
| Irinotecan | Nucleic | Nucleic | Nucleic | Nucleic | Nucleic |
| | acids + | acids + | acids + | acids + | acids + |
| | lysosome | lysosome | lysosome | lysosome | lysosome |
| Cefpiramide | Nucleic | Nucleic | Nucleic | Nucleic | Nucleic |
| | acids + | acids + | acids + | acids + | acids + |
| | lysosome | lysosome | lysosome | lysosome | lysosome |
| Ceftazidime | Nucleic | Nucleic | Nucleic | Nucleic | Nucleic |
| | acids + | acids + | acids + | acids + | acids + |
| | lysosome | lysosome | lysosome | lysosome | lysosome |
| Cefixime | Nucleic | Nucleic | Nucleic | Nucleic | Nucleic |
| | acids + | acids + | acids + | acids + | acids + |
| | lysosome | lysosome | lysosome | lysosome | lysosome |
| Cefotaxime | Nucleic | Nucleic | Nucleic | Nucleic | Nucleic |
| | acids + | acids + | acids + | acids + | acids + |
| | lysosome | lysosome | lysosome | lysosome | lysosome |
| Cefmenoxime | Nucleic | Nucleic | Nucleic | Nucleic | Nucleic |
| | acids + | acids + | acids + | acids + | acids + |
| | lysosome | lysosome | lysosome | lysosome | lysosome |
| Bromocriptine | Nucleic | Nucleic | Nucleic | Nucleic | Nucleic |
| | acids + | acids + | acids + | acids + | acids + |
| | lysosome | lysosome | lysosome | lysosome | lysosome |
| Ergotamine | Nucleic | Nucleic | Nucleic | Nucleic | Nucleic |
| | acids + | acids + | acids + | acids + | acids + |
| | lysosome | lysosome | lysosome | lysosome | lysosome |
| Dihydroer- | Nucleic | Nucleic | Nucleic | Nucleic | Nucleic |
| gotamine | acids + | acids + | acids + | acids + | acids + |
| | lysosome | lysosome | lysosome | lysosome | lysosome |
| Reserpine | Nucleic | Nucleic | Nucleic | Nucleic | Nucleic |
| | acids + | acids + | acids + | acids + | acids + |
| | lysosome | lysosome | lysosome | lysosome | lysosome |
| Deserpine | Nucleic | Nucleic | Nucleic | Nucleic | Nucleic |
| | acids + | acids + | acids + | acids + | acids + |
| | lysosome | lysosome | lysosome | lysosome | lysosome |
| Rifabutin | Nucleic | Nucleic | Nucleic | Nucleic | Nucleic |
| | acids + | acids + | acids + | acids + | acids + |
| | lysosome | lysosome | lysosome | lysosome | lysosome |
| Naldemedine | Nucleic | Nucleic | Nucleic | Nucleic | Nucleic |
| | acids + | acids + | acids + | acids + | acids + |
| | lysosome | lysosome | lysosome | lysosome | lysosome |
| Oxaprozin | Nucleic | Nucleic | Nucleic | Nucleic | Nucleic |
| | acids + | acids + | acids + | acids + | acids + |
| | lysosome | lysosome | lysosome | lysosome | lysosome |
| Benzethonium | Nucleic | Nucleic | Nucleic | Nucleic | Nucleic |
| | acids + | acids + | acids + | acids + | acids + |
| | lysosome | lysosome | lysosome | lysosome | lysosome |
| Rescinnamine | Nucleic | Nucleic | Nucleic | Nucleic | Nucleic |
| | acids + | acids + | acids + | acids + | acids + |
| | lysosome | lysosome | lysosome | lysosome | lysosome |
| Lymecycline | Nucleic | Nucleic | Nucleic | Nucleic | Nucleic |
| | acids + | acids + | acids + | acids + | acids + |
| | lysosome | lysosome | lysosome | lysosome | lysosome |
| Moxifloxacin | Nucleic | Nucleic | Nucleic | Nucleic | Nucleic |
| | acids + | acids + | acids + | acids + | acids + |
| | lysosome | lysosome | lysosome | lysosome | lysosome |
| Dactinomycin | Nucleic | Nucleic | Nucleic | Nucleic | Nucleic |
| | acids + | acids + | acids + | acids + | acids + |
| | lysosome | lysosome | lysosome | lysosome | lysosome |
| Ertapenem | Nucleic | Nucleic | Nucleic | Nucleic | Nucleic |
| | acids + | acids + | acids + | acids + | acids + |
| | lysosome | lysosome | lysosome | lysosome | lysosome |

Table 7 below shows non-limiting examples of optical biopsy stain panels according to the present invention. The stain panels listed below use a combination of nucleus dyes and protein/cytoplasm dyes that have been optimized for their compatibility.

| | | Approximate | Nucleus dyes | | | | |
|---|---|---|---|---|---|---|---|
| | | peak | | Methylene | | | Pyrvinium |
| | | fluorescence | Daunorubicin | Blue | Mitoxantrone | Proflavine | Pamoate |
| | | (nm) | 550, 590 | 695 | 685 | 508 | 700 |
| Protein/ | Fluorescein | 505 | X | X | X | | X |
| cytoplasm | Indigo | 400, 450 | X | X | X | X | X |
| dyes | Carmine | | | | | | |
| | Phloxine B | 550 | | X | X | | X |
| | Phenol Red | 535 | | X | X | | X |
| | Rose Bengal | 565 | | X | X | X | X |
| | Congo Red | 425 | X | X | X | X | X |

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. An optical biopsy stain panel comprising two or more agents, either of which fluorescently stains:
a. nucleic acids;
b. cytoplasm;
c. cell or subcellular membranes;
d. subcellular organelles;
e. extracellular matrix components; or
f. microbes.

2. The stain panel of claim 1, wherein one or more of the agents are clinical drugs or therapeutic drugs.

3.-4. (canceled)

5. The stain panel of claim 1, wherein the two or more agents are existing clinically-used agents, wherein at least one agent is a fluorescent nucleic acid stain selected from a group of nucleic acid-binding agents consisting of: aminoacridines, anthracyclines, anthracenediones, or phenothiazines.

6. The stain panel of claim 5, wherein the aminoacridines are 9-aminmoacridine or proflavine.

7. The stain panel of claim 5, wherein the anthracyclines are Daunorubicin, Doxorubicin, Epirubicin, Idarubicin, Nogalamycin, or Valrubicin.

8. The stain panel of claim 5, wherein the anthracenediones are Mitoxantrone or Pixantrone.

9. The stain panel of claim 5, wherein the phenothiazine is Methylene Blue.

10. The stain panel of claim 1, wherein the two or more agents are existing clinically-used agents, wherein at least one agent is a fluorescent cytoplasmic stain selected from a group of non-specific protein binding agents consisting of: heterotetracyclic and heteropentacyclic compounds; xanthylium dyes; azo dyes; indoles, or phthalein dyes.

11. The stain panel of claim 10, wherein the heterotetracyclic and heteropentacyclic compounds are fluorescein or Rose Bengal.

12. The stain panel of claim 10, wherein the xanthylium dye is phloxine B.

13. The stain panel of claim 10, wherein the azo dye is Congo Red.

14. The stain panel of claim 10, wherein the indole is Indigo Carmine.

15. The stain panel of claim 10, wherein the phthalein dye is phenol red.

16. The stain panel of claim 1, wherein the two or more agents are existing clinically-used agents, wherein at least one of the agents is a fluorescent cell stain or subcellular membrane stain selected from a group of lipid-binding agents consisting of (proto)porphyrins, ace inhibitors, adrenergics/dopaminergics, antihyperglycemics, antineoplastics, bacitracins, carbapenems, cefalosporins, cyclooxygenase inhibitors, indigotindisulfonic acid, kinase inhibitors, Na+ channel blockers, non-dihydropyridine calcium channel blockers, opioids, phosphodiesterase inhibitors, rifamycins, ristocetins, and tetracyclines.

17. The stain panel of claim 1, wherein the two or more agents are existing clinically-used agents, wherein at least one of the agents is a fluorescent organelle stain selected from a group of organelle binding agents consisting of antimitotics, opioids, restocins, rifamycins, porphyrins, atypical antihistamines, cephalosporins, anti-insomnia agents, anti-inflammatory agents, antineoplastics, antifungal agents, hematopoietic stem cell mobilizers, H1 antagonists, MAOIs, class 1 antiarrythmics, antiviral agents, topoisomerase inhibitors, cephalosporins, dopaminergics/adrenergics, non-steroidal anti-inflammatory agents, and broad-spectrum antimicrobial agents.

18. The stain panel of claim 1, wherein the two or more agents are existing clinically-used agents, wherein at least one of the agents is an extracellular matrix stain.

19. The stain panel of claim 1, wherein the two or more agents are existing clinically-used agents, wherein at least one of the agents is a microbial stain.

20. The stain panel of claim 1, wherein the two or more agents comprise a fluorescent nucleic acid stain and a fluorescent cytoplasmic stain, wherein the fluorescent nucleic acid stain is Daunorucibin Methylene Blue, Mitoxantrone, or Proflavine, and the fluorescent cytoplasmic stain is Fluorescein, Phenol Red, Phloxine B, or Rose Bengal.

21.-29. (canceled)

30. An apparatus where the illumination of an optical biopsy stain panel according to claim 1 is about a single wavelength, wherein observation occurs through color filtering means or amplification or conversion of the resultant fluorescence into observable color range.

31.-34. (canceled)
